(19) United States
(12) Patent Application Publication
(10) Pub. No.: US 2008/0028491 A1
(43) Pub. Date: Jan. 31, 2008

(54) Hybrids of Exacum

(76) Inventor: Andrew Riseman, Vancouver (CA)

Correspondence Address: COOLEY GODWARD KRONISH LLP;ATTN: Patent Group, Suite 1100, 777 - 6th Street, NW, WASHINGTON, DC, 20001, US

(21) Appl. No.: 11/494,757

(22) Filed: Jul. 28, 2006

(51) Int. Cl. A01H 5/00; C12N 5/04

(52) U.S. Cl. 800/295; 435/419

(57) ABSTRACT

An Exacum hybrid, method to produce the hybrid plant, and progeny derived from the Exacum hybrid are disclosed. The Exacum hybrid is derived from an interspecific cross with Exacum taxa E. pedunculatum, E. macranthum, E. pallidum, E. trinervium and E. trinervium ssp. ritigalensis native to Sri Lanka. Also disclosed is a cell culture comprising regenerable cells obtained from the Exacum hybrid, a method of producing an Exacum hybrid from the regenerable cells, and an Exacum hybrid produced from the regenerable cells.

### FIELD OF INVENTION

The present invention relates to plants of the genus Exacum. More specifically, the present invention provides interspecific hybrid populations of Exacum derived from several Sri Lankan Exacum taxa having desirable traits for commercial floriculture.

### BACKGROUND OF THE INVENTION

The genus Exacum L. (Gentianaceae Juss.) contains approximately 65 species, most of which are annual or perennial herbs (Klackenberg, J (1985) Opera Botanica pp. 1-144. AiO Print Ltd. Copenhagen). Of the recognized species, only E. affine L., native to Socotra, has been successfully domesticated and introduced to commercial floriculture. More than 25 cultivars of E. affine L. are commercially available with the most popular being ‘Royal Dane Blue’.

In the system proposed by the Angiosperm Phylogeny Group (Bremer et al., (1998) Ann. Missouri Bot. Gard. 85: 531-553) Exacum is classified among the Asterids. Furthermore, Gentianales (Order) and Gentianaceae (Family) groups are supported and deemed valid. Below this level, the International Association for Plant Taxonomy has accepted the following scheme for Exacum (Reveal (1995) Indices nominum supragenericorum plantarum vascularium (see: matrix.nal.usda.gov:8080/star/supragenericname.html)

| Subfamily | Gentianoideae |
|---|---|
| Tribe | Exaceae |
| Subtribe | Exacinae |
| Genus | Exacum |

Serveral species and taxa native to Sri Lanka have been identified as a potential new horticulture crop based on the presence of horticulturally desirable traits (Riseman, A. and R. Craig (1995) Acta Horticulturae, 420:132-134. XVIIIth EUCARPIA Symposium Section Ornamentals, Tel Aviv, Israel. 5-9 Mar. 1995). These traits include prolific flowering during summer; abundant, large flowers (4 to 7 cm in diameter) that range in color from light blue to dark violet with contrasting bright yellow anthers; diverse growth habits from erect to decumbent; attractive, glossy green foliage; and efficient production attributes, for example easily propagation via cutting. However, no single species or taxon possesses sufficient desirable traits for either direct introduction or domestication. For example, E. macranthum Arn., known primarily for its large, dark blue-violet flowers, was identified over 30 years ago as a potential new horticultural crop. Several independent breeding programs using this species as the sole germplasm source have failed to produce a commercially viable cultivar. Despite garnering significant attention for beautiful flowers, failure to produce a worthy introduction was attributed to the lack of genetic variation of those non-floral traits important for horticultural acceptance, for example height, branching habit and garden performance.

### SUMMARY OF THE INVENTION

The present invention relates to plants of the genus Exacum. More specifically, the present invention provides interspecific hybrid populations of Exacum derived from several Sri Lankan Exacum taxa having desirable traits for commercial floriculture.

It is an object of the present invention to provide improved hybrid plants of the genus Exacum.

The present invention provides Exacum hybrids derived from an interspecific crosses with Exacum taxa E. pedunculatum, E. macranthum, E. pallidum, E. trinervium and E. trinervium ssp. ritigalensis native to Sri Lanka. The hybrids are characterized as having a flowering time of about 80 to about 150 days, zinc efficient, a height of about 10 to about 30 cm, a leaf length of about 4 to about 13 cm, a leaf width of about 1.5 to about 4.0 cm, a flower size of about 2 to about 8 cm, a branching density of about 2 to 3 per 5 cm over the length of the stem, and a vertical growth habit. The present invention also pertains to any progeny derived from the Exacum hybrids as just defined.

The present invention also provides an Exacum hybrid that is characterized as comprising a stem shape that is cylindrical to quadrangular, with, or without wings or lines, with a height at anthesis of about 12 to about 36 cm, a leaf shape that is lanceolate to ovate or narrowly elliptic with a length from about 5.5 to about 11 cm, and a width from about 1.8 to about 3.7 cm width, a flower colour that is pale blue to dark violet and characterized with a Royal Horticultural Society of England (RHS) score of 89B-C through to 93A-C, a calyx lobe length of about 3 to about 24 mm, a petal apex arrangement that is rhomboidal to broadly obovate, overlapping or not, with a length of about 20 to about 28 cm and a width of about 16 to about 21 mm, and an anther length of about 8 to about 18 mm. The present invention also pertains to progeny derived from the Exacum hybrids as just defined.

The present invention provides an Exacum hybrid grex with representative seed of the variety to be deposited within the ATCC, and to progeny derived from the Exacum plants as just defined. Examples of the Exacum hybrid grex include, but are not limited to the following representative varieties: 01-09-01, 01-37-08, 01-37-61, 01-42-03, 01-47-21, 01-47-47, 01-50-46, 01-69-17, 01-48-10, and 02-174-09.

This invention also relates to tissue cultures of regenerable cells from the hybrid Exacum plants described above. The tissue culture will preferably be capable of regenerating plants having physiological and morphological characteristics comprising a flowering time of about 80 to about 150 days, zinc efficient, a height of about 10 to about 30 cm, a leaf length of about 4 to about 13 cm, a leaf width of about 1.5 to about 4.0 cm, a flower size of about 2 to about 8 cm, a branching density of about 2 to about 3 nodes per 5 cm over the length of the stem, and a vertical growth habit, and of regenerating plants having substantially the same genotype as the foregoing Exacum plant. Preferably, the regenerable cells in such tissue cultures are embryos, protoplasts, seeds, callus, pollen, leaves, anthers, roots, and meristematic cells. Still further, the present invention provides Exacum plants regenerated from the tissue cultures of the invention, as well as to the use of the tissue cultures for regenerating Exacum plants. The present invention also pertains to progeny derived from the Exacum plants as just defined.

This invention further relates to the parts of the Exacum plants described above, including their cells, pollen, ovules, roots, leaves, seeds, microspores and vegetative parts, whether mature or embryonic. It also relates to the use of these plant parts for regenerating a Exacum characterized as having a flowering time of about 80 to about 150 days, zinc efficient, a height of about 10 to about 30 cm, a leaf length of about 4 to about 13 cm, a leaf width of about 1.5 to about 4.0 cm, a flower size of about 2 to about 8 cm, a branching density of about 2 to about 3 per 5 cm over the length of the stem, and a vertical growth habit, and to the plants regenerated therefrom.

The present invention relates to the use of the plants described above for breeding a Exacum line, through pedigree breeding, crossing, self-pollination, haploidy, single seed descent, modified single seed descent, and backcrossing, or other suitable breeding methods, and to the plants produced therefrom.

This invention also provides a method for producing a hybrid Exacum seed by crossing one of the plants described above with an inbred Exacum plant of a different variety or species, and harvesting the resultant first generation (F1) hybrid Exacum seed. It further relates to the plants produced from the F1 hybrid seed.

The present invention also provides for a method for producing a hybrid Exacum by interspecific cross with Exacum taxa E. pedunculatum, E. macranthum, E. pallidum, E. trinervium and E. trinervium ssp. ritigalensis native to Sri Lanka, the hybrids characterized as having a flowering time of about 80 to about 150 days, zinc efficient, a height of about 10 to about 30 cm, a leaf length of about 4 to about 13 cm, a leaf width of about 1.5 to about 4.0 cm, a flower size of about 2 to about 8 cm, a branching density of about 2 to about 3 per 5 cm over the length of the stem, and a vertical growth habit. Furthermore, this invention includes an Exacum hybrid produced using the method as just described, and to progeny derived from the Exacum hybrid.

This invention provides substantial value to commercial producers by providing hitherto unavailable combinations of including an hybrid Exacum characterized as having a flowering time of about 80 to about 150 days, zinc efficient, a height of about 10 to about 30 cm, a leaf length of about 4 to about 13 cm, a leaf width of about 1.5 to about 4.0 cm, a flower size of about 2 to about 8 cm, a branching density of about 2 to about 3 per 5 cm over the length of the stem, and a vertical growth habit. This trait combination provides hybrid Exacum plants that are horticulturally acceptable and suited for commercial production.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a crossing diagram representing production of viable hybrids from five selected Sri Lankan Exacum taxa. Arrow heads point to the taxon used as the seed parent in the cross. Double-headed arrows indicate successful reciprocal crosses. The thickness of each arrow represents the number of successful primary interspecific hybrids, wherein:

[symbol] =production of many primary interspecific hybrids (>50)

[symbol] =production of some primary interspecific hybrids (10 to 50)

[symbol] =production of few primary interspecific hybrids (<10)

FIG. 2 shows a flowchart representing breeding selection of viable hybrids from five Sri Lankan Exacum taxa, namely E. pedunculatum L., E. macranthum Arn., E. pallidum Trimen., E. trinervium (L.) Druce and E. trinervium ssp. ritigalensis (Willis) Cramer. FIG. 2a shows the predigree for the selection genotype 01-09-01; FIG. 2b shows the breeding selection for selected genotypes 01-37-08/01-37-37/01-37-61; FIG. 2c shows the predigree for the selection genotype 01-42-03; FIG. 2d shows the predigree for the selection genotypes 01-47-21/01-47-49; FIG. 2e shows the predigree for the selection genotype 01-48-10; FIG. 2f shows the predigree for the selection genotype 01-50-46; FIG. 2g shows the predigree for the selection genotype 01-69-17; and FIG. 2h shows the predigree for the selection genotype 02-174-09. Each plant in the breeding program shown in FIGS. 2a-2h is assigned a unique genotype identification (ID) consisting of three numbers. The first number is the year the seed was sown; the second number is the family code (i.e., specific parents in a specific combination) by convention, seed parent (female) is listed first followed by pollen parent (male); and the third number is the individual sibling from that family. When multiple individuals from a family are selected their IDs are separated by a ‘/’. Since they are siblings, they have the same pedigree. In reading the pedigree, arrows indicate a new generation and there are two choices for arrow placement in FIGS. 2a-2h:
1. Arrow tail is at a ‘x’ separating two genotype IDs and the arrowhead points to a family genotype ID. This indicates that the family was produced by that combination of parents; or
2. Arrow tail begins at a family genotype ID and the arrowhead is also to a family genotype ID. This indicates the plants are the same individual. This is also the case when an arrow is pointing toward a family genotype ID and originating at a species name; they are the same entity.

FIG. 3 shows a photograph of hybrid Exacum population segregating for zinc efficiency. Zinc efficient plants are indicated by a thick solid, open circle [symbol] and inefficient plants are indicated by multiple thin open circles [symbol] .

FIG. 4 shows comparative photos of available Exacum plants and an example of an hybrid Exacum of the present invention. FIG. 4a, shows E. affine ‘Midget White’ and ‘Midget Blue’ (Sakata); FIG. 4b, shows E. affine ‘Blue Champion’ (E. J. Small); FIG. 4c shows E. affine ‘Royal Dane’ series (Ex-Plant AsP); FIG. 4d shows an example of interspecific Exacum hybrid of the present invention variety 1-48-10; FIG. 4e shows a range of Exacum Styer group, displaying plant habits from 12^{th} generation hybrids (ruler showing both inch and cm units); and FIG. 4f shows a range of flowers obtained from individual 12^{th} generation hybrids of the Exacum Styer group (ruler showing both inch and cm units).

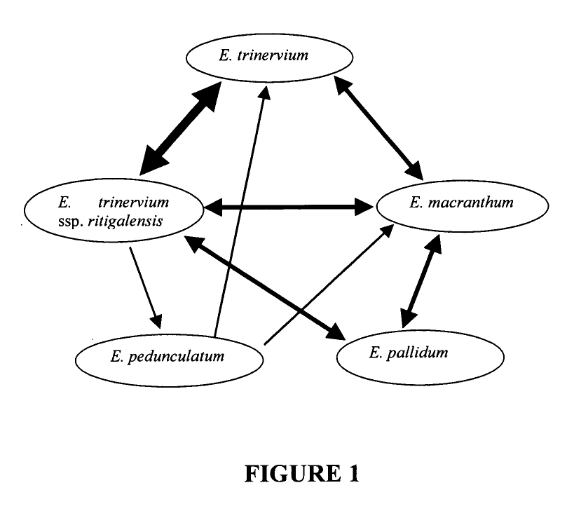

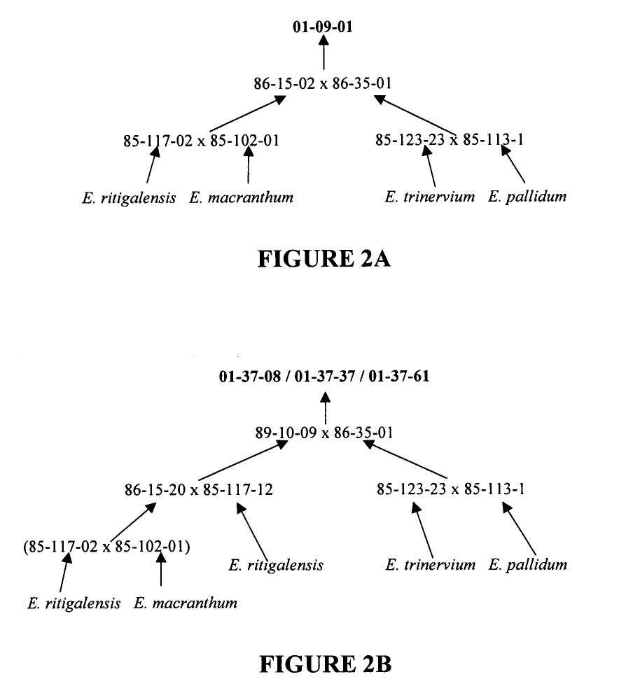

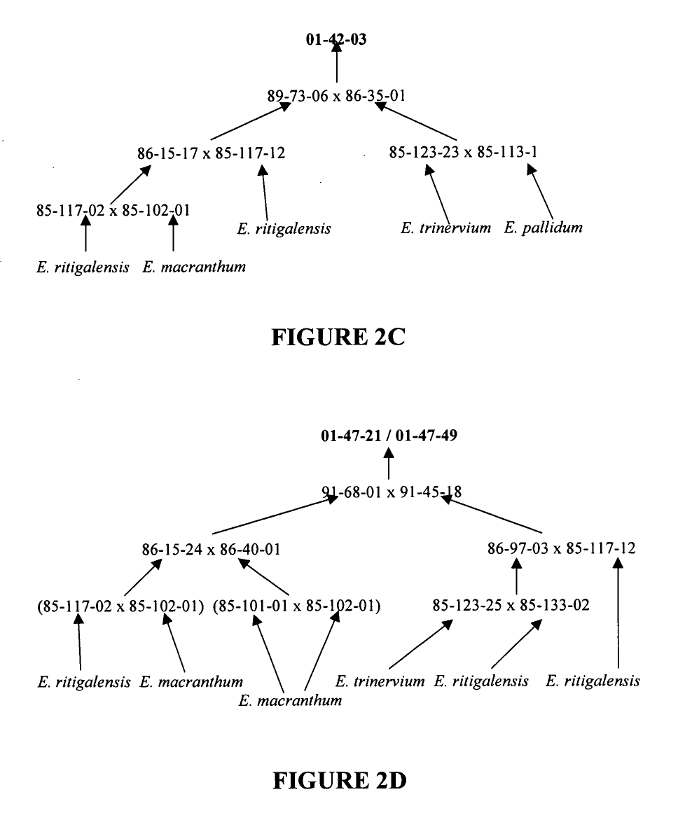

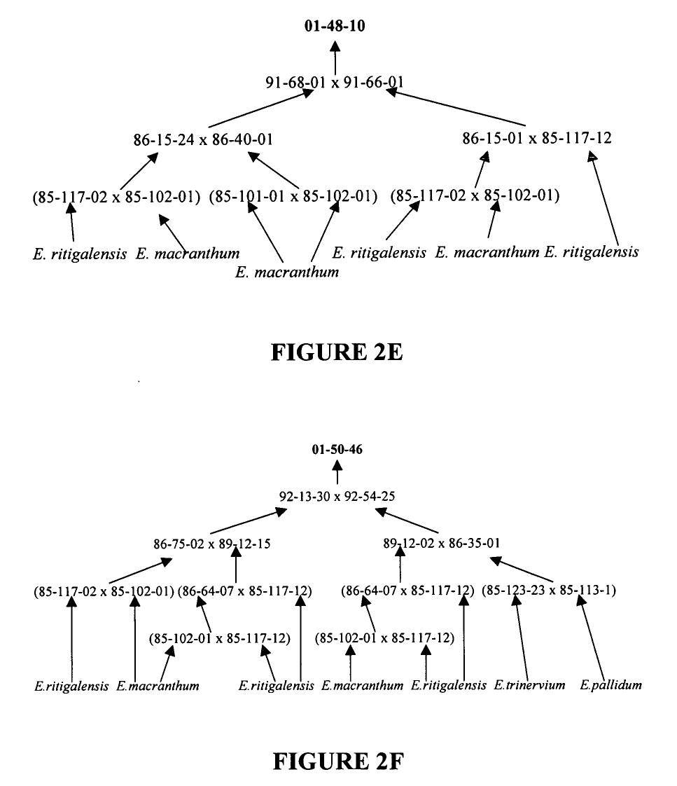

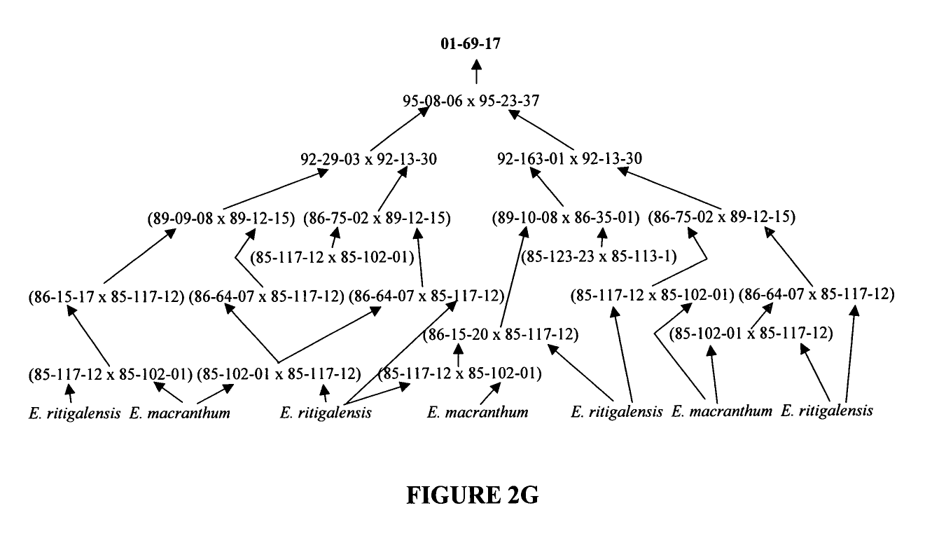

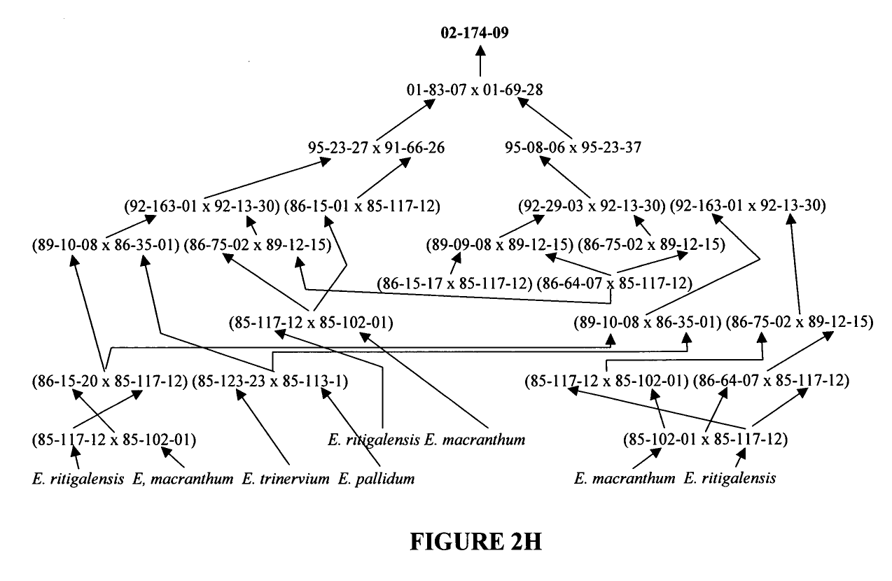

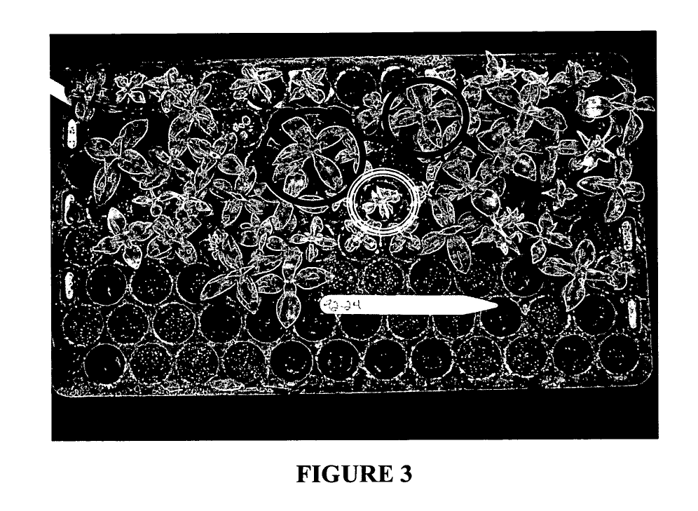

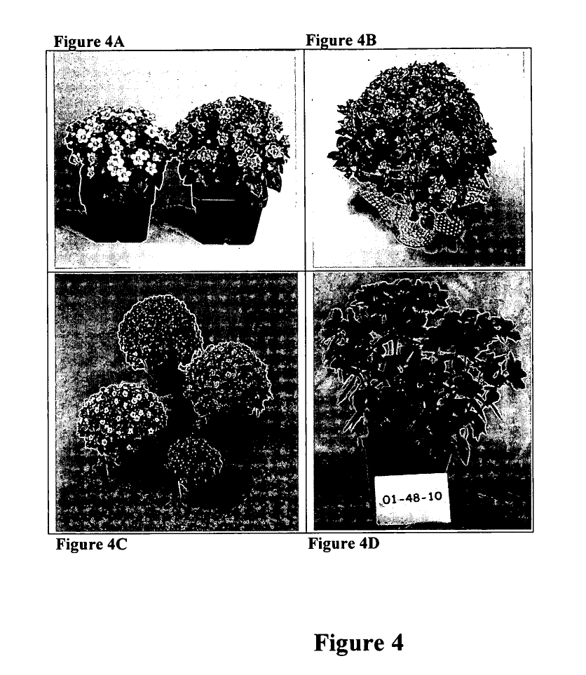

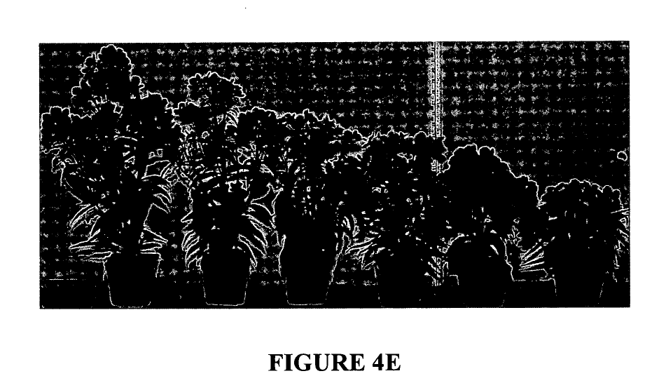

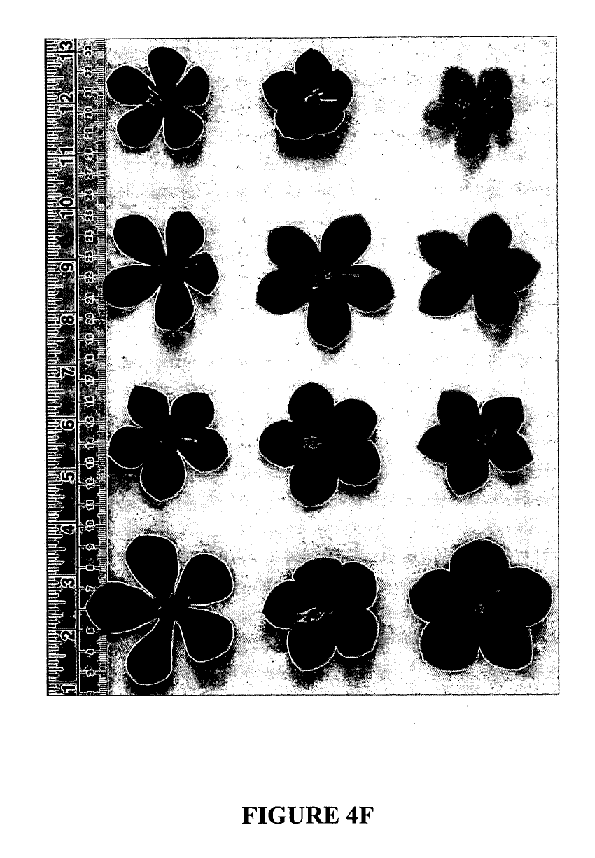

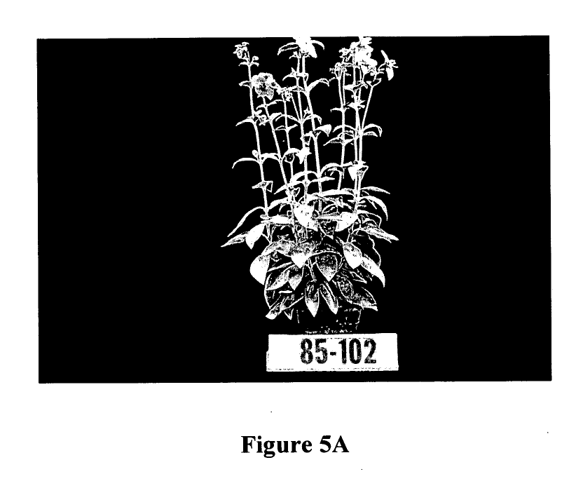

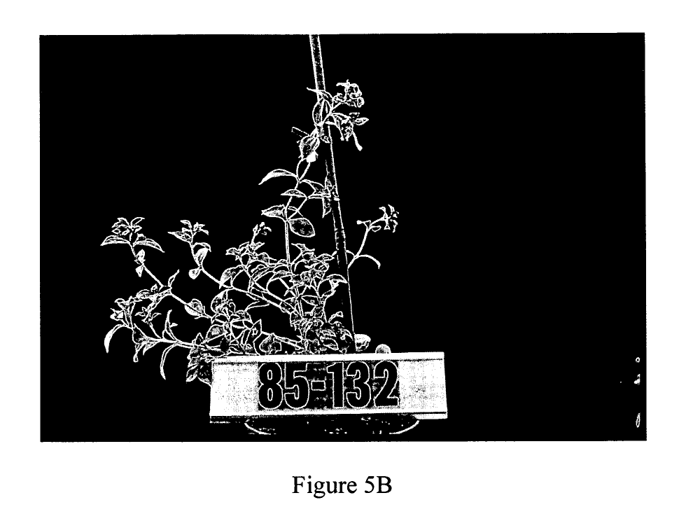

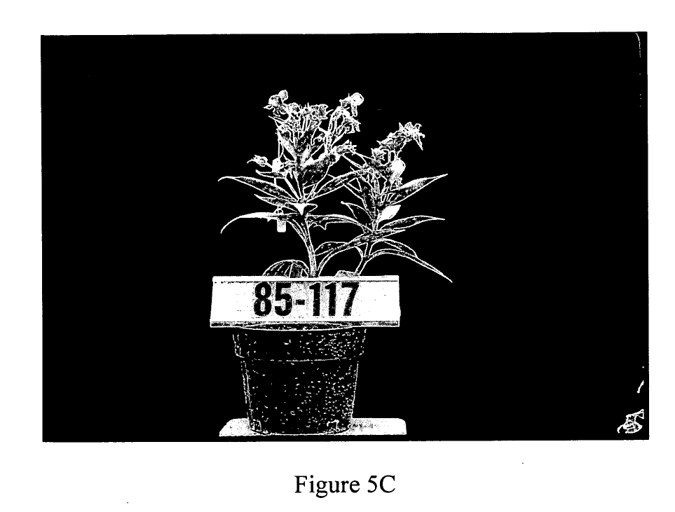

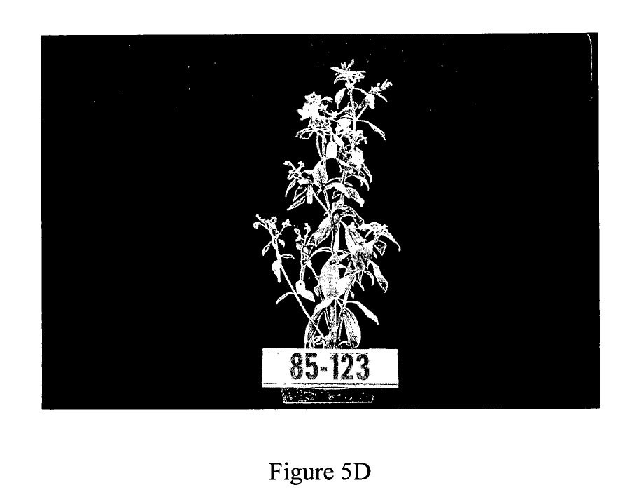

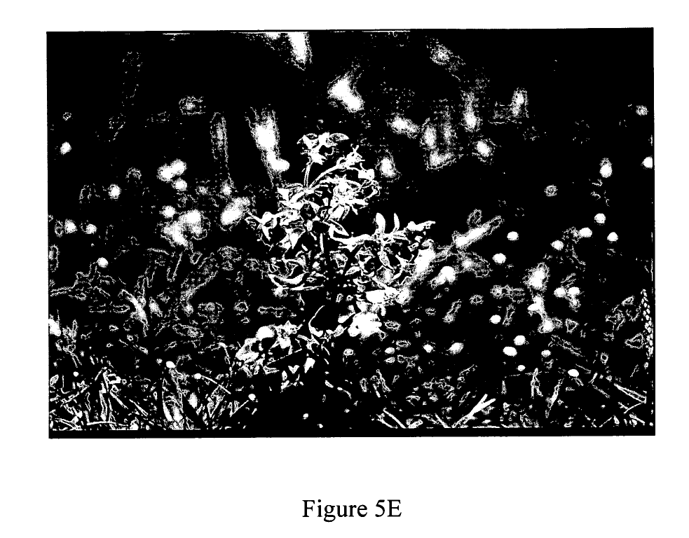

FIG. 5 shows photos of Exacum plants identified in FIG. 1. FIG. 5a shows greenhouse produced Exacum macranthum; FIG. 5b shows greenhouse produced Exacum pallidum; FIG. 5c shows greenhouse produced Exacum pedunculatum; FIG. 5d shows greenhouse produced Exacum trinervium ssp ritigalensis; FIG. 5e shows Exacum pedunculatum native in Sri Lanka.

### DETAILED DESCRIPTION

The present invention relates to plants of the genus Exacum. More specifically, the present invention provides interspecific hybrid populations of Exacum derived from several Sri Lankan Exacum taxa having desirable traits for commercial floriculture.

The following description is of a preferred embodiment.

As described in more detail below, Exacum hybrids were derived from an interspecific crosses with Exacum taxa E. pedunculatum, E. macranthum, E. pallidum, E. trinervium and E. trinervium ssp. ritigalensis native to Sri Lanka (see FIGS. 1). The hybrid plants are characterized as having a flowering time of about 80 to about 150 days, zinc efficient, a height of about 10 to about 30 cm (see FIG. 4e), a leaf length of about 4 to about 13 cm, a leaf width of about 1.5 to about 4.0 cm, a flower size of about 2 to about 8 cm (see FIG. 4f), a branching density of about 2 to about 3 per 5 cm over the length of the stem, and a vertical growth habit. Examples of representative plants, which are not to be considered limiting in any manner are shown in FIGS. 4d and 4e, and described in more detail in tables 1-3 below.

The hybrid Exacum plants of the present invention are more easily produced, and characterized as having increased hardiness, are of a reduced plant height, and that are more easily cultivated than the parental lines (identified in FIG. 1), and therefore these plants are horticulturally acceptable and suited for commercial production. Examples of the parental Exacum lines used for the breeding of the hybrid plants of the present invention are shown in FIGS. 5a-e. An example of interspecific hybrids derived from the Exacum native to Sri Lanka as described in the present invention is, Exacum Styer Group (see FIG. 4e for the range in plant habits for the group; Riseman A et al. 2005, Hort Sci 40:1580-1583, which is incorporated herein by reference). Generally, members of the grex of the present invention, “Exacum Styer Group”, are characterized as comprising a stem shape that is cylindrical to quadrangular, with, or without wings or lines (height at anthesis: 12-36 cm); leaf shape that is lanceolate to ovate or narrowly elliptic (5.5-11×1.8-2.7 cm—L×W); flower colour that is pale blue to dark violet (Royal Horticultural Society of England (RHS) 89B-C through 93A-C; 5-merous); calyx lobe length of 3-24 mm (see FIG. 4f); petal apex arrangement rhomboidal to broadly obovate, overlapping or not (20-28×16-21 mm; L×W; see FIG. 4f); and an anther length of 8-18 mm. Progeny of the Exacum hybrid plants as described herein are also included within the scope of the present invention.

Plant habit or growth habit refers to the form of the plant, for example a plant can be erect (upright), where at least 80% of one or more than one main stem is within about ±10° from perpendicular; leaning, where at least 80% of the one or more than one main stem is about ±20° to about 45° from perpendicular; or sprawling, where about 80% of the one or more than one main stem is procumbent.

Plant height is a measure taken from the pot edge to the top of the plant canopy and is measured in centimeters. Plant height is variable and age dependant. Further, plant height may be dependant upon a range of criteria including growth conditions, for example, light intensity, temperature, humidity, fertilization regime, addition of growth regulators, and zinc supplementation. Therefore plant height may be manipulated by varying one or more of these variables. Plant height may reach up to about 60 cm, however, a plant height of about 10 cm to about 30 cm, or any amount therebetween is typically a desired range in plant height.

Flowering of the Exacum hybrids of the present invention may also be varied. The onset of flowering may be delayed under conditions of low light, by growing a taller plant, by adding exogenous compounds, for example, ethreyl (an ethyl-ethyl dimmer), or a combination thereof.

Hardiness. Hardiness refers to the US National Arboretum (USDA) Plant Hardiness Zone Map, USDA Miscellaneous Publication No. 1475 Issued January 1990, available from U.S. National Arboretum, Agricultural Research Service, U.S. Department of Agriculture, Washington, D.C. 20002. Zone 11 represents any area where the average annual minimum temperature is above 40 F (4.4 C).

Stem branching or branching density is a measure of the number of branches emerging from, or nodes associated with a branch within a defined length of one or more than one main stem when measured over the length of the stem, or in the top 50% of plant height. For example, a branching density of 3 per 5 cm over the length of the main stem, indicates that along the length of the stem, on average there will be 3 branches per 5 cm. Alternatively, within any 5 cm length of stem in the upper half of the plant, approx. 3 branches will emerge from the one or more than one main stem.

“RHS” refers to the Royal Horticultural Society of England official botanical color chart quantitatively identifying colors according to a defined numbering system. The chart is available from Royal Hort Society Enterprise Ltd RHS Garden; Wisley, Woking; Surrey GU236QB, UK.

By increased hardiness, it is meant that the plants are tolerant to a range of environmental factors when compared to the parental plant lines, including but not limited to increased tolerance to Fusarium infection, zinc, and pot culture. Parental plant lines exhibit poor growth when potted and grown under greenhouse conditions. However, the plants of the present invention are readily produced under greenhouse growth conditions.

Resistance to a disease is when about 80% to about 100% of the plants in a given population do not show any symptoms of the disease through a production cycle. Preferably, 90% to about 100% of the plants do not show any symptoms of the disease through a production cycle. By tolerance to a disease it is meant that the disease may still grow on the plant, but that the plant does not display any negative effects, for example which is not to be considered limiting, plant biomass may not reduced, disease progression may be inhibited, or both.

Zinc efficiency is the ability of a genotype to grow normally under low zinc availability conditions. In the case of the parental Sri Lanka Exacum species (FIGS. 1 and 5a-5e) and their progeny (for example as outlined in FIGS. 2b-2h), the amount of zinc present in standard fertilizers is too low for growth, and these plants exhibit symptoms of zinc deficiency (Riseman A and Craig R., 2000, Plant and Soil 219:41-47, is incorporated herein by reference). Zn deficiency is also observed in the parental Sri Lanka Exacum species and plants derived from the parental lines, when grown under high-light conditions as found in the greenhouse. The Exacum hybrids of the present invention, or the parental cultivars therefore require above 4 μM solution of Zn applied biweekly to supplement normal fertilization.

By symptoms of zinc deficiency it is meant the appearance of any one or more than one of the following deficiency symptoms: chlorosis, rosette formation, longitudinal leaf curl, and necrosis. A zinc inefficient Exacum plant exhibits an uptake of Zn of less than about 6.5 μmol Zn/cm root surface, for example from about 1.0 to about 6.4 μmol Zn/cm root surface or any amount therebetween. A zinc efficient Exacum plant exhibits an uptake of Zn of greater than or equal to about 6.5 μmol Zn/cm root surface, for example from about 6.5 to about 20 μmol Zn/cm root surface or any amount therebetween, or about 6.7 to about 12.5 μmol Zn/cm root surface, or any amount therebetween. Other parameters may also be used to determine zinc efficiency/inefficiency including zinc uptake/cm root length, or μmol Zn/mg root as disclosed in Riseman and Craig (2000, Plant and Soil 219:41-47). Zinc efficiency may also be defined in terms of frequency within a population, for example, a Zn inefficient plant cultivar may comprise from about 80 to about 100% of a population of plants, or any amount therebetween, and require zinc supplementation as described above.

An example of conditions for the growth of the Exacum plants of the present invention include:
Light: 18 hr Photoperiod; Intensity: minimum of 800 μmol m⁻² sec⁻¹ through either ambient light or ambient light supplemented with high intensity lamps
Temperature: 20-24° C. Day, 18-20° C. night
Media: standard peat-lite commercial greenhouse mix
Fertilization: constant feed of 21-7-7 Acid fertilizer at 100-200 ppm total nitrogen (or another high ammonium:nitrate ratio fertilizer).
pH: 4-6
EC (Electrical conductivity): 1-2.25 mS cm⁻¹

The Exacum plants of the present invention were produced through repeated cycles of selection, the selected genotypes include all the distinguishing traits identified in the original Sri Lankan Exacum population shown in Table 1. However, as a result of initial interspecific hybridizations and subsequent breeding, these traits are expressed in novel combinations and as intermediate forms not displayed in the original taxa. The selected genotypes display full sexual compatibility.

More particularly the selected genotypes of the present invention are characterized as having the following traits:

Plants are procumbent to erect having stems that are cylindrical to quadrangular, with wings present or lacking;

Leaves are glossy green, lanceolate to ovate or narrowly elliptic;

Leaf tips are acute to acuminate and leaf bases cuneate to rounded; and

Flowers are blue to violet with pale to dark hues. The flowers are flattened to cup-shaped or rarely with petals reflexed with the corolla base short and tubular. The petals are rhomboidial to broadly obovate, and overlapping along their entire length to overlapping only at their broadest point or not overlapping. The anthers are bright yellow and 8 to 18 mm long.

Seeds from examples of plants obtained as described herein include but are not limited to genotypes 01-09-01, 01-37-08, 01-37-61, 01-42-03, 01-47-21, 01-47-47, 01-48-10, 01-50-46, 01-69-17, 02-174-09 as described in Tables 2a-2d, below. Representative seed of this grex to be deposited in the ATCC.

TABLE 1
Distinguishing morphological characters of Sri Lankan Exacum species (see FIGS. 5a–5e).

| | Stem Shape | | Flower Color | Calyx | Petal Apex | |
|---|---|---|---|---|---|---|
| | (Height at | Leaf Shape | RHS* code | Lobe | arrangement | Anther |
| Species | anthesis) | (Length × Width) | (petal number) | Length | (Length × Width) | Length |
| E. macranthum | Cylindrical | Ovate to oval- | Dark violet | 8–9 mm | Acuminate overlapping | 6–13 mm |
| | (29–55 cm) | suborbicular | RHS 89B | | (14–32 × 8–25 mm) | |
| | | (4.5–9.7 × 1.8–4.3 cm) | (5-merous) | | | |
| E. pallidum | Cylindrical with | Ovate | Pale blue | 9–10 mm | Acute not overlapping | 2–4 mm |
| | 2 opposite lines | (3.0–7.5 × 1.6–2.3 cm) | RHS 91C | | 10–11 × 6–8 mm | |
| | (17–48 cm) | | (5-merous) | | | |
| E. | Quadrangular | Elliptic to ovate | Medium blue | 3–4 mm | Acute, reflexed | 4 mm |
| pedunculatum | (8–22 cm) | (1–2 × 0.5–0.6 cm) | RHS 90D | | overlapping | |
| | | | (4-merous) | | 6–8 × 4 mm, | |
| E. trinervium | Quadrangular | Lanceolate to narrowly | Medium to light blue | 7–10 mm | Acute not overlapping | 6–13 mm |
| | with wings | elliptic | RHS 92B | | 6–16 × 5–15 mm | |
| | (39–73 cm) | (5.6–11.8 × 1.8–4.7 cm) | (5-merous) | | | |
| E. trinervium | Quadrangular | Lanceolate to ovate or | Medium to dark blue | 10–24 mm | Acute overlapping | 6–13 mm |
| ssp. ritigalensis | with wings | narrowly elliptic | RHS 93B–D | | 6–25 × 6–20 mm | |
| | (20–63 cm) | (6.5–14.8 × 1.3–4.3 cm) | (5-merous) | | | |

*RHS code is in accordance with the RHS Color Chart of the Royal Horticulture Society (RHS), London, England (1998)

Examples of characteristics of genotypes 01-09-01, 01-37-08 and 01-37-61, obtained as shown in FIGS. 2a and 2b, are presented in Table 2a. However, it is to be understood that the characteristics listed in Table 2a are not to be construed as limiting in any manner, but rather as they are provided examples of the features obtained.

TABLE 2a
Distinguishing traits for genotypes 01-09-01, 01-37-08, and 01-37-61

| | 01-09-01 | 01-37-08 | 01-37-61 |
|---|---|---|---|
| Trait | | | |
| Botanical classification: | Gentianaceae | Gentianaceae | Gentianaceae |
| Commercial classification: | Annual | Annual | Annual |
| Use: | Ornamental | Ornamental | Ornamental |
| Parentage: | 86-15-02 × 86-35-01 | 89-10-09 × 86-35-01 | 89-10-09 × 86-35-01 |
| Plant Description: | | | |
| Bloom Period.- | Spring/Summer | Spring/Summer | Spring/Summer |
| Plant Habit.- | Erect. | Erect. | Erect. |
| Height from pot lip.- | 31 cm. | 31 cm. | 28.5 cm. |
| Width.- | 32.5 cm. | 36 cm. | 39.0 cm. |
| Hardiness.- | USDA Zone 11 | USDA Zone 11 | USDA Zone 11 |
| Propagation.- | Vegetative Cuttings | Vegetative Cuttings | Vegetative Cuttings |
| Time to develop roots.- | 21–28 Days | 21–28 Days | 21–28 Days |
| Crop time.- | 12–14 weeks | 12–14 weeks | 12–14 weeks |
| Stem: | | | |
| Shape.- | Square. | Square. | Square. |
| Stem color.- | RHS 144A. | RHS 144A. | RHS 144A. |
| Stem dimensions.- | 21 cm in height | 21 cm in height | 17.0 cm in height |
| | 3 mm in diameter. | 5 mm in diameter. | 3.5 mm in diameter. |
| Stem surface.- | Glabrous; glandular. | Glabrous; glandular. | Glabrous; glandular. |
| Basal stem shape.- | Square. | Square. | Square. |
| Basal stem color.- | RHS 144A. | RHS 144A. | RHS 144A. |
| Branching.- | Free Branching | Free Branching | Free Branching |
| Internode length.- | 1.5 cm–4 cm | 2.5 cm–3.5 cm | 1.5 cm–3 cm |
| Branching habit.- | Upright | Upright | Upright |
| Foliage: | | | |
| Type.- | Evergreen. | Evergreen. | Evergreen. |
| Shape.- | Elliptic - lanceolate | Elliptic - lanceolate | Elliptic - lanceolate |
| Division.- | Simple | Simple | Simple |
| Apex.- | Apiculate - acuminate | Apiculate - | Apiculate -acuminate. |
| | | acuminate. | |
| Base.- | Attenuate - cuneate. | Attenuate - cuneate. | Attenuate - cuneate. |
| Venation.- | Parallel | Parallel | Parallel |
| Vein color (adaxial | RHS 146B | RHS 144B | RHS 146C |
| surfaces).- | | | |
| Vein color (abaxial | RHS 144A | RHS 144B | RHS 144B |
| surfaces).- | | | |
| Margins.- | Entire | Entire | Entire |
| Texture.- | Smooth; glabrous | Smooth; glabrous | Smooth; glabrous |
| Arrangement.- | Opposite | Opposite | Opposite |
| Attachment.- | Sessile | Sessile | Sessile |
| Surfaces (adaxial and | Smooth; glandular | Smooth; glandular | Smooth; glandular |
| abaxial).- | (abaxial) | (abaxial) | (abaxial) |
| Mature leaf dimensions.- | 5.5 cm × 1.8 cm | 11 cm × 3.5 cm | 9 cm × 3.5 cm |
| Young leaf dimensions.- | 1.2 cm × 2.5 mm | 2.8 cm × 9 mm | 1.9 cm × 9 mm |
| Leaf color (adaxial | RHS 147B | RHS 147A | RHS 146A |
| surface).- | | | |
| Leaf color (abaxial | RHS 146A | RHS 147B | RHS 146A |
| surface).- | | | |
| Petiole shape.- | Sessile | Sessile | Sessile |
| Flowers: | | | |
| Inflorescence type.- | Compound cyme | Compound cyme | Compound cyme |
| Dimensions of | 33.5 cm × 14.5 cm | 29 cm × 15 cm | 37.5 cm × 14.5 cm |
| inflorescence.- | | | |
| Shape.- | Actinomorphic | Actinomorphic | Actinomorphic; rotate |
| Flower dimensions.- | 4.2 cm × 2.3 cm | 5.7 cm × 2.8 cm | 5 cm × 2.6 cm |
| Persistent or self- | Persistent | Persistent | Persistent |
| cleaning.- | | | |
| Aspect.- | Facing upward | Facing upward | Facing upward |
| Sexuality.- | Bisexual | Bisexual | Bisexual |
| Bud dimensions.- | 1.1 cm × 7 mm | 7 mm × 1.6 cm | 9 mm × 1.4 cm |
| Bud shape.- | Oval; winged | Oval; winged | Oval; winged |
| Bud color.- | RHS 144A | RHS 144A | RHS 144A |
| Flower color.- | RHS 89C | RHS 93A | RHS 93B |
| Petals.- | Five | Five | Five |
| Fused or unfused.- | Unfused | Unfused | Unfused |
| Petal dimensions.- | 2 cm × 1.7 cm | 2.8 cm × 2 cm | 2.5 cm × 1.9 cm |
| Petal margin.- | Entire | Entire | Entire |
| Petal surface.- | Glabrous | Glabrous | Glabrous |
| Calyx dimensions.- | 1.0 cm × 8.0 mm | 1.1 cm × 8 mm | 1.1 cm × 9 mm |
| Calyx surface.- | Glabrous; glandular | Glabrous; glandular | Glabrous; glandular |
| Sepals.- | Five | Five | Five |
| Sepals fused or unfused | Unfused | Unfused | Unfused |
| Sepal margin.- | Entire; sinuate | Entire; sinuate | Entire; sinuate |
| Sepal color.- | RHS 147B | RHS 137C | RHS 137C |
| Sepal surface.- | Glabrous; glandular | Glabrous; glandular | Glabrous; glandular |
| Sepal apex.- | Ligulate - apiculate | Ligulate - apiculate | Ligulate - apiculate |
| Peduncle dimensions.- | 7.5 mm × 5 mm | 9 mm × 6 mm | 8 mm × 7 mm |
| Peduncle color.- | RHS 144C | RHS 144B | RHS 144B |
| Peduncle surface.- | Glabrous; glandular | Glabrous; glandular | Glabrous; glandular |
| Reproductive organs: | | | |
| Stamens.- | Five | Five | Five |
| Stamen color.- | RHS 14A | RHS 14B | RHS 14B |
| Stamen dimensions.- | 1.6 cm × 2 mm | 1.8 cm × 2 mm | 1.9 cm × 1.5 mm |
| Pistil.- | One | One | One |
| Pistil dimensions.- | 1.6 cm × 1 mm in | 1.9 cm × 1 mm | 2 cm × 1 mm |
| | diameter. | | |
| Pistil color.- | RHS 144C | RHS 144C | RHS 144C |
| Ovary position.- | Superior; stamens | Superior; stamens | Superior; stamens |
| | perigynous. | perigynous. | perigynous. |
| Ovary color.- | 144A | 144A | 144A |
| Ovary shape.- | Oval | Oval | Oval |
| Ovary dimensions.- | 7 mm × 3 mm | 6 mm × 2.5 mm | 7 mm × 3 mm |

Table 2b shows the traits for selected genotype 01-42-03, 01-47-21, and 01-47-21 obtained as shown in FIGS. 2C and 2D

TABLE 2b
Distinguishing traits for genotypes 01-42-03, 01-47-21, and 01-47-49

| | 01-42-03 | 01-47-21 | 01-47-49 |
|---|---|---|---|
| Trait | | | |
| Botanical classification: | Gentianaceae | Gentianaceae | Gentianaceae |
| Commercial classification: | Annual | Annual | Annual |
| Use: | Ornamental | Ornamental | Ornamental |
| Parentage: | 89-73-06 × 86-35-01 | 91-68-01 × 91-45-18 | 91-68-01 × 91-45-18 |
| Plant Description: | | | |
| Bloom Period.- | Spring/Summer | Spring/Summer | Spring/Summer |
| Plant Habit.- | Erect. | Erect. | Erect. |
| Height from pot lip.- | 36 cm. | 28 cm. | 23 cm. |
| Width.- | 36 cm. | 36 cm. | 30 cm. |
| Hardiness.- | USDA Zone 11 | USDA Zone 11 | USDA Zone 11 |
| Propagation.- | Vegetative Cuttings | Vegetative Cuttings | Vegetative Cuttings |
| Time to develop roots.- | 21–28 Days | 21–28 Days | 21–28 Days |
| Crop time.- | 12–14 weeks | 12–14 weeks | 12–14 weeks |
| Stem: | | | |
| Shape.- | Square. | Square. | Square. |
| Stem color.- | RHS 144A. | RHS 144A. | RHS 144A. |
| Stem dimensions.- | 27 cm in height and | 20 cm in height and | 13 cm in height and |
| | 3 mm in diameter. | 3 mm in diameter. | 4 mm in diameter. |
| Stem surface.- | Glabrous; glandular. | Glabrous; glandular. | Glabrous; glandular. |
| Basal stem shape.- | Square. | Square. | Square. |
| Basal stem color.- | RHS 144A. | RHS 144A. | RHS 144A. |
| Branching.- | Free Branching | Free Branching | Free Branching |
| Internode length.- | 4 cm | 2.0 cm–2.5 cm | 1.8 cm–6 cm |
| Branching habit.- | Upright | Upright | Upright |
| Foliage: | | | |
| Type.- | Evergreen. | Evergreen. | Evergreen. |
| Shape.- | Elliptic - lanceolate. | Elliptic - lanceolate. | Lanceolate. |
| Division.- | Simple. | Simple. | Simple. |
| Apex.- | Apiculate - acuminate. | Apiculate - acuminate. | Acuminate. |
| Base.- | Attenuate - cuneate. | Attenuate - cuneate. | Attenuate - cuneate. |
| Venation.- | Parallel | Parallel | Parallel |
| Vein color (adaxial | RHS 144B | RHS 146A | RHS 146B |
| surfaces).- | | | |
| Vein color (abaxial | RHS 144C | RHS 144B | RHS 144A |
| surfaces).- | | | |
| Margins.- | Entire | Entire | Entire |
| Texture.- | Smooth; glabrous | Smooth; glabrous | Smooth; glabrous |
| Arrangement.- | Opposite | Opposite | Opposite |
| Attachment.- | Sessile | Sessile | Sessile |
| Surfaces (adaxial and | Smooth; glandular | Smooth; glandular | Smooth; glandular |
| abaxial).- | (abaxial) | (abaxial) | (abaxial) |
| Mature leaf dimensions.- | 7.9 cm × 3.7 cm | 7.6 cm × 2.6 cm | 9.2 cm × 3 cm |
| Young leaf dimensions.- | 2.7 cm × 1.8 cm | 2.4 cm × 1.1 cm | 4.2 cm × 1.2 cm |
| Leaf color (adaxial | RHS 147A | RHS 147A | RHS 147A |
| surface).- | | | |
| Leaf color (abaxial | RHS 147B | RHS 147B | RHS 146A |
| surface).- | | | |
| Petiole shape.- | Sessile | Sessile | Sessile |
| Flowers: | | | |
| Inflorescence type.- | Compound cyme | Compound cyme | Compound cyme |
| Dimensions of | 36 cm × 14.5 cm | 38 cm × 10 cm | 35 cm × 11 cm |
| inflorescence.- | | | |
| Shape.- | Actinomorphic; rotate | Actinomorphic; rotate | Actinomorphic |
| Flower dimensions.- | 4.3 cm × 2.3 cm | 4.3 cm × 2.2 cm | 4.1 cm × 2 cm |
| Persistent or self- | Persistent | Persistent | Persistent |
| cleaning.- | | | |
| Aspect.- | Facing upward | Facing upward | Facing upward |
| Sexuality.- | Bisexual | Bisexual | Bisexual |
| Bud dimensions.- | 7 mm × 1.1 cm | 8 mm × 1.2 cm | 9 mm × 1.3 cm |
| Bud shape.- | Oval; winged | Oval; winged | Oval; winged |
| Bud color.- | RHS 144A | RHS 144A | RHS 144A |
| Flower color.- | RHS 93B | RHS 93C | RHS 93C |
| Petals.- | Five | Five | Five |
| Fused or unfused.- | Unfused | Unfused | Unfused |
| Petal dimensions.- | 2.2 cm × 1.9 cm | 2.2 cm × 1.9 cm | 2 cm × 1.6 cm |
| Petal margin.- | Entire | Entire | Entire |
| Petal surface.- | Glabrous | Glabrous | Glabrous |
| Calyx dimensions.- | 1.0 cm × 9 mm | 1.2 cm × 1.1 cm | 1.1 cm × 1 cm |
| Calyx surface.- | Glabrous; glandular | Glabrous; glandular | Glabrous; glandular |
| Sepals.- | Five | Five | Five |
| Sepals fused or unfused | Unfused | Unfused | Unfused |
| Sepal margin.- | Entire; sinuate | Entire; sinuate | Entire; sinuate |
| Sepal color.- | RHS 137C | RHS 137C | RHS 137B |
| Sepal surface.- | Glabrous; glandular | Glabrous; glandular | Glabrous; glandular |
| Sepal apex.- | Ligulate - apiculate | Ligulate - apiculate | Ligulate - apiculate |
| Peduncle dimensions.- | 7 mm × 7 mm | 1.2 cm × 6 mm | 7 mm × 5 mm |
| Peduncle color.- | RHS 144A | RHS 144B | RHS 144C |
| Peduncle surface.- | Glabrous; glandular | Glabrous; glandular | Glabrous; glandular |
| Reproductive organs: | | | |
| Stamens.- | Five | Five | Five |
| Stamen color.- | RHS 7A | RHS 14B | RHS 153B |
| Stamen dimensions.- | 1.5 cm × 1.5 mm | 1.5 cm × 2 mm | 1.3 cm × 1.5 mm |
| Pistil.- | One | One | One |
| Pistil dimensions.- | 1.6 cm × 0.75 mm | 1.5 cm × 1 mm | 1.2 cm × 1 mm |
| Pistil color.- | RHS 144B | RHS 144C | RHS 144B |
| Ovary position.- | Superior; stamens | Superior; stamens | Superior; stamens |
| | perigynous. | perigynous. | perigynous. |
| Ovary color.- | RHS 144A | RHS 144A | RHS 144A |
| Ovary shape.- | Oval | Oval | Oval |
| Ovary dimensions.- | 6 mm × 3 mm | 7 mm × 2.5 mm | 4 mm × 2.5 mm |

Table 2c shows the traits for selected genotype 01-48-10 (obtained as shown in FIG. 2E); selected genotype 01-50-46 (obtained as shown in FIG. 2F); and selected genotype 01-69-17 (obtained as shown in FIG. 2G).

TABLE 2c
Distinguishing traits for genotypes 01-48-10, 01-50-46, and 01-69-17

| | 01-48-10 | 01-50-46 | 01-69-17 |
|---|---|---|---|
| Trait | | | |
| Botanical classification: | Gentianaceae | Gentianaceae | Gentianaceae |
| Commercial classification: | Annual | Annual | Annual |
| Use: | Ornamental | Ornamental | Ornamental |
| Parentage: | 91-68-01 × 91-66-01 | 92-13-30 × 92-54-25 | 95-08-06 × 95-23-37 |
| Plant Description: | | | |
| Bloom Period.- | Spring/Summer | Spring/Summer | Spring/Summer |
| Plant Habit.- | Erect. | Erect. | Erect. |
| Height from pot lip.- | 22.5 cm. | 26 cm. | 21 cm. |
| Width.- | 29 cm. | 34 cm. | 29 cm. |
| Hardiness.- | USDA Zone 11 | USDA Zone 11 | USDA Zone 11 |
| Propagation.- | Vegetative Cuttings | Vegetative Cuttings | Vegetative Cuttings |
| Time to develop roots.- | 21–28 Days | 21–28 Days | 21–28 Days |
| Crop time.- | 12–14 weeks | 12–14 weeks | 12–14 weeks |
| Stem: | | | |
| Shape.- | Square. | Square. | Square. |
| Stem color.- | RHS 144B. | RHS 144A. | RHS 144A. |
| Stem dimensions.- | 16.5 cm in height and | 14 cm in height and | 16 cm in height and |
| | 3 mm in diameter. | 2.5 mm in diameter. | 3 mm in diameter. |
| Stem surface.- | Glabrous; glandular. | Glabrous; glandular. | Glabrous; glandular. |
| Basal stem shape.- | Square. | Square. | Square. |
| Basal stem color.- | RHS 144A. | RHS 144A. | RHS 144A. |
| Branching.- | Free Branching | Free Branching | Free Branching |
| Internode length.- | 2.7 cm–4.3 cm | 2.2 cm–3.2 cm | 2.8 cm |
| Branching habit.- | Upright | Upright | Upright |
| Foliage: | | | |
| Type.- | Evergreen. | Evergreen. | Evergreen. |
| Shape.- | Lanceolate - elliptic. | Elliptic - lanceolate. | Elliptic - lanceolate. |
| Division.- | Simple. | Simple. | Simple. |
| Apex.- | Apiculate - acuminate. | Apiculate - acuminate. | Apiculate - acuminate. |
| Base.- | Attenuate - cuneate. | Attenuate - cuneate. | Attenuate - cuneate. |
| Venation.- | Parallel | Parallel | Parallel |
| Vein color (adaxial | | RHS 146B | RHS 146C |
| surfaces).- | | | |
| Vein color (abaxial | RHS 144B | RHS 146C | RHS 144B |
| surfaces).- | | | |
| Margins.- | Entire | Entire | Entire |
| Texture.- | Smooth; glabrous | Smooth; glabrous | Smooth; glabrous |
| Arrangement.- | Opposite | Opposite | Opposite |
| Attachment.- | Sessile | Sessile | Sessile |
| Surfaces (adaxial and | Smooth; glandular | Smooth; glandular | Smooth; glandular |
| abaxial).- | (abaxial) | (abaxial) | (abaxial) |
| Mature leaf dimensions.- | 9 cm × 3 cm | 6.7 cm × 2.7 cm | 7 cm × 2.4 cm |
| Young leaf dimensions.- | 3.7 cm × 1.1 cm | 2.3 cm × 7 mm | 1.7 cm × 6 mm |
| Leaf color (adaxial | RHS 147A | RHS 147A | RHS 147A |
| surface).- | | | |
| Leaf color (abaxial | RHS 147B | RHS 146A | RHS 147B |
| surface).- | | | |
| Petiole shape.- | Sessile | Sessile | Sessile |
| Flowers: | | | |
| Inflorescence type.- | Compound cyme | Compound cyme | Compound cyme |
| Dimensions of | 31.5 cm × 12.5 cm | 34 cm × 17 cm | 27 cm × 15 cm |
| inflorescence.- | | | |
| Shape.- | Actinomorphic; rotate | Actinomorphic | Actinomorphic |
| Flower dimensions.- | 5.3 cm × 2.6 cm | 5.7 cm × 2.1 cm | 4.5 cm × 2.2 cm |
| Persistent or self- | Persistent | Persistent | Persistent |
| cleaning.- | | | |
| Aspect.- | Facing upward | Facing upward | Facing upward |
| Sexuality.- | Bisexual | Bisexual | Bisexual |
| Bud dimensions.- | 6 mm × 1.1 cm | 7 mm × 1.4 cm | 1.0 cm × 1.4 cm |
| Bud shape.- | Oval; winged | Oval; winged | Oval; winged |
| Bud color.- | RHS 144A | RHS 144A | RHS 144A |
| Flower color.- | RHS 93B | RHS 93B | RHS 89B |
| Petals.- | Five | Five | Five |
| Fused or unfused.- | Unfused | Unfused | Unfused |
| Petal dimensions.- | 2.6 cm × 2.1 cm | 2.8 cm × 1.7 cm | 2.3 cm × 1.8 cm |
| Petal margin.- | Entire | Entire | Entire |
| Petal surface.- | Glabrous | Glabrous | Glabrous |
| Calyx dimensions.- | 1.2 cm × 1.1 cm | 1.1 cm × 8 mm | 1.4 cm × 1.0 cm |
| Calyx surface.- | Glabrous; glandular | Glabrous; glandular | Glabrous; glandular |
| Sepals.- | Five | Five | Five |
| Sepals fused or unfused. | Unfused | Unfused | Unfused |
| Sepal margin.- | Entire; sinuate | Entire; sinuate | Entire; sinuate |
| Sepal color.- | RHS 137B | RHS 137A | RHS 137C |
| Sepal surface.- | Glabrous; glandular | Glabrous; glandular | Glabrous; glandular |
| Sepal apex.- | Ligulate - apiculate | Ligulate - apiculate | Ligulate - apiculate |
| Peduncle dimensions.- | 9 mm × 8 mm | 7 mm × 5 mm | 8 mm × 8 mm |
| Peduncle color.- | RHS 137B | RHS 144C | RHS 137B |
| Peduncle surface.- | Glabrous; glandular | Glabrous; glandular | Glabrous; glandular |
| Reproductive organs: | | | |
| Stamens.- | Five | Five | Five |
| Stamen color.- | RHS 14B | RHS 14B | RHS 14A |
| Stamen dimensions.- | 1.8 cm × 2 mm | 1.5 cm × 2 mm | 1.7 cm × 1 mm |
| Pistil.- | One | One | One |
| Pistil dimensions.- | 1.8 cm × 1 mm | 1.8 cm × 1 mm | 2 cm × 1 mm |
| Pistil color.- | RHS 144C | RHS 144C | RHS 144B |
| Ovary position.- | Superior; stamens | Superior; stamens | Superior; stamens |
| | perigynous. | perigynous. | perigynous. |
| Ovary color.- | RHS 144A | RHS 144A | RHS 144A |
| Ovary shape.- | Oval | Oval | Oval |
| Ovary dimensions.- | 7 mm × 3.5 mm | 7 mm × 2.5 mm | 8 mm × 3 mm |

Genotype 02-174-09 (see FIG. 2h) is characterized in Table 2d

TABLE 2d
Distinguishing traits for genotype 02-174-09

Botanical classification: Gentianaceae.
Plant Description:
Plant Habit. - Erect.
Height from pot lip. - 29.5 cm.
Width. - 26 cm.
Disease problems. - Fusarium solani.
Stem:
Shape. - Square with wings.
Stem color. - 144B.
Stem dimensions. - 27 cm in height and 13 mm in diameter.
Stem surface. - Glabrous
Basal stem shape. - Square with wings
Basal stem color. - 144B
Branching. - Naturally branched
Internode length. - 3.1 cm between nodes.
Branching habit. - Mid to upper branching with erect stems.
Foliage:
Type. - Evergreen.
Shape. - Elliptic - lanceolate.
Division. - Simple.
Apex. - Apiculate - Acuminate.
Base. - Attenuate - cuneate.
Venation. - Parallel with veins (3) depressed on adaxial surface and
protruding on abaxial surface.
Vein color (adaxial surfaces). - 146B.
Vein color (abaxial surfaces). - 145B.
Margins. - Entire.
Texture. - Smooth; glabrous.
Arrangement. - Opposite.
Attachment. - Sessile.
Surfaces (adaxial and abaxial). - Smooth, glandular for both surfaces
Mature leaf dimensions. - 11.5 cm in length and 4.3 cm in width at
widest point.
Young leaf dimensions. - 2.7 cm in length and 8 mm in width.
Leaf color (adaxial surface). - 147A.
Leaf color (abaxial surface). - Center is 147B. Margin is 146B.
Petiole shape. - Sessile.
Petiole color. - 146D
Petiole surface. - Glabrous
Petiole dimensions. - 5 mm width
Flowers:
Inflorescence type. - Compound cyme.
Dimensions of inflorescence. - 12 cm in width and 8.5 cm in height.
Shape. - Actinomorphic. rotate
Flower dimensions. - 5.7 cm in width and 2.4 cm in height.
Persistent or self-cleaning. - Persistent.
Aspect. - Facing upwards.
Sexuality. - Bisexual.
Bud dimensions. - 7.5 mm in width and 2.2 cm in length.
Bud shape. - Oval; winged.
Bud color. - 145C.
Flower color. - 95A
Petals. - Five in number.
Fused or unfused. - Unfused.
Petal dimensions. - 2.8 cm in length and 2.6 cm in width at widest part.
Petal margin. - Entire.
Petal surface. - Glabrous.
Calyx dimensions. - 1.4 cm in height and 1.8 cm in diameter.
Calyx surface. - Glabrous
Sepals. - Five in number.
Sepals fused or unfused. - Unfused.
Sepal margin. - Entire
Sepal color. - 143A
Sepal surface. - Glabrous
Sepal apex. - Ligulate - apiculate.
Peduncle dimensions. - 3.0 cm in length and 7 mm in diameter.
Peduncle color. - 143B
Peduncle surface. - Glabrous
Reproductive organs:
Stamens. - Five in number.
Stamen color. - 3B
Stamen dimensions. - 1.8 cm in length and 2 mm in diameter.
Pistil. - One.
Pistil dimensions. - 2.7 cm in length and 1 mm in diameter.
Pistil color. - 144D
Ovary position. - Superior; stamens perigynous.
Ovary color. - 144A.
Ovary shape. - Oval.
Ovary dimensions. - 8 mm in height and 8 mm in diameter.

Each of genotypes 01-09-01, 01-37-08, 01-37-61, 01-42-03, 01-47-21, 01-47-47, 01-48-10, 01-50-46, 01-69-17, or 02-174-09 is suitable for horticultural production.

### Asexual Reproduction

All plant genotypes can be asexually propagated by stem cuttings. However, higher rooting percentages are typically observed when cuttings are harvested from non-flowering stock plants. For stem cuttings of Exacum, cuttings 2-3 nodes in length are typically basally treated with 1% IBA (Hormodin #1, MSD-AGVET, Division of Merck and Co., NJ) in talc, or 10 mM NAA solution for 20 seconds. The cuttings can then be inserted into cell packs filled with a well-drained substrate (for example a peat-based potting mix) and placed under intermittent mist until rooted. Rooted cuttings are generally obtained within 2-4 weeks and rooting percentages typically range from 50% to 100% depending on genotype and reproductive status of the stock plant. Rooted cuttings are generally transplanted into 10-12 cm pots with a well-drained peat based media (pH 4.5-5.5).

Hybrid Exacum plants may also be propagated by tissue culture using regenerable cells and methods that are well known to one of skill in the art (for example Riesman and Chennareddy, 2004, J. Ameri. Soc. Hort. Sci. 129:698-703, which is incorporated herein by reference). Regenerable cells include embryos, protoplasts, seeds, callus, pollen, leaves, anthers, roots, and meristematic cells.

The present invention includes a cell culture comprising regenerable cells obtained from the Exacum hybrid as defined herein, and Exacum plants regenerated from these tissue cultures. This invention also provides a method of producing an Exacum hybrid involving, providing regenerable cells, and growing the Exacum hybrid from the regenerable cells.

### Growth Medium and Fertilization

An acidifying fertilizer high in nitrogen, such as Plant-Prod 21-7-7, is suitable used at a rate of 150-200 ppm total nitrogen. The medium pH may be managed through the use of fertilizer; a higher ammonium:nitrate ratio helped to maintain a low medium pH. The optimal media pH range is typically 4.0 to 6.0, or any amount therebetween, for example, pH 5.0-6.0, and the optimal medium electrical conductivity (EC) between 1 to 2.25 dS·cm⁻¹. Plants were treated with zinc sulfate at 10 ppm every two weeks to prevent the development of zinc stress during seed production.

### Irrigation

Plants generally perform best when grown on the ‘dry side’ but without undo wilting. In general, plants seemed more tolerant of drought than overly wet conditions. Typically, excessive water or a severe drought followed by excessive water leads to increased susceptibility to root pathogens. Also, plant growth is generally enhanced by periodically wetting the foliage with either water or a dilute fertilizer solution.

### Pest Management

Greenhouse whitefly (Trialeurodes vaporariorum) may be controlled through application of several commercial pesticides. However, Malathion® is shown to have phytotoxic effects on mature buds and open flowers. Fungus gnats (Bradysia ssp. Winnertz) may be controlled through introduction of parasitic nematodes or by application of a Bti (Bacillus thuringiensis Berliner spp. israelensis) based product. In addition, the root pathogens Pythium Pringsh., Phytophthora deBary., and Fusarium Link ex Fr. may be controlled though a combination of cultural modification (e.g., less frequent irrigations) and media drenches with commercial fungicides.

### Environmental Conditions and Flowering Time

Aerial temperature is ideally maintained between 18-26° C. day and 16-20° C. night and relative humidity maintained between 70-85% throughout the production cycle. Photoperiod control is generally not required for flowering.

Flowering time varies based on genotype. The earliest genotypes typically flower 6-8 weeks after transplanting into a finishing container while the latest genotypes flower between 16-20 weeks following transplanting. Flowering time generally decreases with increasing light intensities, however, during naturally low light months, supplemental irradiance may promote earlier and more robust flowering for all genotypes.

The present invention will be further illustrated in the following examples.

### EXAMPLE 1

### Origin of Plants

Five taxa of Exacum were collected from their native habitats in Sri Lanka, namely E. pedunculatum L., E. macranthum Arn., E. pallidum Trimen., E. trinervium (L.) Druce and E. trinervium ssp. ritigalensis (Willis) Cramer. The distinguishing characteristics for each of these five taxa are provided in gtabl1 (above) and briefly as follows:

### E. macranthum

Stems 29-55 cm long, cylindrical in cross-section; basal internodes medium to long. Leaves mainly ovate to oval-suborbicular, seldom elliptical, 4.5-9.7×1.8-4.3 cm; apices broadly acute to broadly acuminate. Flowers dark violet, sometimes with red tinge, 5-merous. Calyx lobes 8-19×1-3 mm. Corolla lobes broadly ovate, obtuse, 14-32×8-25 mm. Anthers 6-13 mm long.

Key characters: Stems terete with no wings or ridges; leaves oval-orbicular with undulating blades.

### E. pallidum

Stems 17-48 cm long, cylindrical with 2 opposite grooves; internodes at base compact to medium. Leaves ovate 3.0-7.5×1.6-2.3 cm; apices broadly acute. Flowers pale blue-violet, 5-merous. Calyx lobes 9-10×1-1.5 mm. Corolla lobes elliptic, 10-11×6-8 mm. Anthers 2-4 mm long, often triangular to oblong.

Key characters: Stems cylindrical with furrows whose alignment alternates with internodes; flowers small, pale blue with short anthers; branching profuse; leaves pale green.

### E. trinervium

Stems 39-73 cm long; basal internodes medium to long. Leaves 3-nerved, elliptical-linear to elliptical-lanceolate, 5.6-11.8×1.8-4.7 cm. Flowers medium to light violet, 5-merous. Calyx lobes 7-17 mm long. Corolla lobes elliptical-ovate, obtuse 6-16×3-15 mm.

Key characters: Slender stems with upright branching.

### E. trinervium Ssp ritigalensis

Stems 20-63 cm long; internodes always quadrangular. Leaves elliptical-ovate to lanceolate-elliptic, 6.5-14.8×1.4-4.3 cm, mostly narrowly acuminate. Flowers medium to dark violet, 5-merous. Calyx lobes 10-24 mm long. Corolla lobes broadly ovate to ovate, 6-25×6-20 mm, always obtuse. Anthers 6-13 mm long.

Key characters: Stems robust, diffusely branching; leaves long, sometime 5-nerved.

The distinguishing traits among the original taxa of Sri Lankan Exacum used in the breeding program are summarized in Table 1.

### Breeding Selection of Hybrid Plants

Primary interspecific hybrids were produced from the crosses as detailed in FIG. 1. These primary hybrids were then used in subsequent breeding, wherein for each generation superior progeny were identified and used as the following generation's parents.

Through all generations, selection was for fertility, greenhouse production traits (e.g., domestication), and combinations of horticulture traits that are indicative of hybrid status. These selection criteria were continued through 12 generations beyond the primary hybrids.

FIG. 2 shows a flowchart representing breeding selection of viable hybrids from the five originally collected Sri Lankan Exacum taxa. FIGS. 2a-2h each represent different selected genotypes resulting from breeding selections of different hybrid plants. The selected genotype is given in bold at the top of each figure and the pedigree works downward.

Each plant in the breeding program was assigned a unique genotype identification (ID) consisting of three numbers. The first number is the year the seed was sown; the second number is the family code (i.e., specific parents in a specific combination) by convention, seed parent (female) is listed first followed by pollen parent (male); and the third number is the individual sibling from that family. When multiple individuals from a family were selected their IDs are separated by a ‘/’. Since they are siblings, they have the same pedigree.

### Culture of Plants

### Hybrid Seed Production

Seed were produced on asexually propagated plants. All families were produced through controlled pollinations under greenhouse conditions. Each pollinated flower was labeled with the seed parent, the pollen parent and the date. Emasculation was not necessary because Exacum pollen is dehisced through apical pores in the anthers which need to be mechanically manipulated for pollen release. In addition, no fruit were ever observed on unpollinated control plants. Fruit were collected at maturity and stored in individual glassine packets until sowing.

### Self-Pollinated Seed Production

Seed were produced on asexually propagated plants. Pollination procedures were as described above. A minimum of 200 pollinations was made for each genotype to ensure adequate seed production.

### Plant Production

Both hybrid and inbred seed and resulting seedlings were treated identically. Seeds were surface sown onto a well drained peat moss based media in individual seed trays with identification labels. Trays were sub-irrigated, allowed to drain, and then enclosed in clear plastic bags. The bagged trays were placed under cool-white fluorescent lamps (GTE, Lighting Division, Salem, Mass.) and maintained at 20 C until germination. Once germination occurred, plastic bags were opened for one day, and then removed. Four weeks after germination, seedlings were transplanted into individual slots in 50 unit plug trays filled with MetroMix 500™ and moved to the greenhouse. All seedlings were maintained in a single greenhouse section in order to minimize environmental variation. Seedlings were sub-irrigated for an additional three weeks until they were large enough to sustain overhead irrigation. Starting at the time of transplanting, and continuing throughout the experiment, seedlings were fertigated with 100 ppm total nitrogen fertilizer supplied by 20-10-20 peat-lite™ (Scotts-Sierra Horticultural Products Co. Marysville, Ohio). Greenhouse conditions were maintained at 20 C night temperature and 25 C day temperature. Plants remained in the plug trays for an additional six weeks and then were transplanted into 10 cm pots filled with the same media. Cultural and environmental conditions were uniform for all plants.

All citations are hereby incorporated by reference.

The present invention has been described with regard to one or more embodiments. However, it will be apparent to persons skilled in the art that a number of variations and modifications can be made without departing from the scope of the invention as defined in the claims.

What is claimed is:

1. An Exacum hybrid derived from an interspecific cross with Exacum taxa E. pedunculatum, E. macranthum, E. pallidum, E. trinervium and E. trinervium ssp. ritigalensis native to Sri Lanka, the hybrids characterized as having a flowering time of about 80 to about 150 days, zinc efficient, a height of about 10 to about 30 cm, a leaf length of about 4 to about 13 cm, a leaf width of about 1.5 to about 4.0 cm, a flower size of about 2 to about 8 cm, a branching density of about 2 to 3 per 5 cm, over the length of the stem, and a vertical growth habit, and progeny derived therefrom.

2. A cell culture comprising regenerable cells obtained from the Exacum hybrid as defined in claim 1.

3. A method of producing an Exacum hybrid comprising providing the regenerable cells of claim 2, and growing the Exacum hybrid from the regenerable cells.

4. An Exacum hybrid produced from the regenerable cells as described in claim 2.

5. An Exacum hybrid produced using the method described in claim 3.

6. A method for producing a hybrid Exacum by interspecific cross with Exacum taxa E. pedunculatum, E. macranthum, E. pallidum, E. trinervium and E. trinervium ssp. ritigalensis native to Sri Lanka, the hybrids characterized as having a flowering time of about 80 to about 150 days, zinc efficient, a height of about 10 to about 30 cm, a leaf length of about 4 to about 13 cm, a leaf width of about 1.5 to about 4.0 cm, a flower size of about 2 to about 8 cm, a branching density of about 2 to 3 per 5 cm over the length of the stem, and a vertical growth habit.

7. The method of claim 6 wherein seed, a stem cutting, or both, is collected from the hybrid Exacum.

8. An Exacum hybrid produced using the method described in claim 6, or progeny therefrom.

9. An Exacum hybrid characterized as comprising a stem shape that is cylindrical to quadrangular, with, or without wings or lines, with a height at anthesis of about 12 to about 36 cm, a leaf shape that is lanceolate to ovate or narrowly elliptic with a length from about 5.5 to about 11 cm, and a width from about 1.8 to about 3.7 cm width, a flower colour that is pale blue to dark violet and characterized with a Royal Horticultural Society of England (RHS) score of 89B-C through to 93A-C, a calyx lobe length of about 3 to about 24 mm, a petal apex arrangement that is rhomboidal to broadly obovate, overlapping or not, with a length of about 20 to about 28 cm and a width of about 16 to about 21 mm, and an anther length of about 8 to about 18 mm, and progeny derived therefrom.

10. A cell culture comprising regenerable cells obtained from the Exacum hybrid as defined in claim 9.

11. A method of producing an Exacum hybrid comprising providing the regenerable cells of claim 10, and growing the Exacum hybrid from the regenerable cells.

12. An Exacum hybrid produced from the regenerable cells as described in claim 10.

13. An Exacum hybrid produced using the method described in claim 11.

14. A method for producing a hybrid Exacum by interspecific cross with Exacum taxa E. pedunculatum, E. macranthum, E. pallidum, E. trinervium and E. trinervium ssp. ritigalensis native to Sri Lanka, the hybrid characterized as having a stem shape that is cylindrical to quadrangular, with, or without wings or lines, with a height at anthesis of about 12 to about 36 cm, a leaf shape that is lanceolate to ovate or narrowly elliptic with a length from about 5.5 to about 11 cm, and a width from about 1.8 to about 3.7 cm width, a flower colour that is pale blue to dark violet and characterized with a Royal Horticultural Society of England (RHS) score of 89B-C through to 93A-C, a calyx lobe length of about 3 to about 24 mm, a petal apex arrangement that is rhomboidal to broadly obovate, overlapping or not, with a length of about 20 to about 28 cm and a width of about 16 to about 21 mm, and an anther length of about 8 to about 18 mm.

15. The method of claim 14 wherein seed, a stem cutting, or both, is collected from the hybrid Exacum.

16. An Exacum hybrid produced using the method described in claim 14, or progeny therefrom.
